(19) United States
(12) Patent Application Publication
(10) Pub. No.: US 2023/0340719 A1
(43) Pub. Date: Oct. 26, 2023

(54) GRAPHENE SILICONE STICKY SHEET AND COMPOUND STRUCTURE USING THE SAME.

(71) Applicant: HAO YANG YANG, Taipei City (TW)

(72) Inventor: HAO YANG YANG, Taipei City (TW)

(21) Appl. No.: 17/725,577

(22) Filed: Apr. 21, 2022

(51) Int. Cl. H05B 3/34; D01F 11/10; D06M 10/06; H05B 3/10

(52) U.S. Cl. CPC D06M 10/06; D01F 11/10; H05B 3/10; H05B 3/345; H05B 2203/036

(57) ABSTRACT

A graphene silicone sticky sheet and compound structure using the same is provided. The graphene silicone with PU function group is used as substrate which is further combined with an adhering layer so as to form as a graphene silicone sticky sheet. Therefore, it is adhered to various application objects, especially clothes or wearing objects, such as an eye mask. Further, the graphene silicone can be combined with conductive wires which extend outwards and thus it can be made as electrode adhesive sheet or electric heating sheet.

### FIELD OF THE INVENTION

The present invention is related to graphene silicone, and in particular to a graphene silicone sticky sheet and compound structure using the same.

### BACKGROUND OF THE INVENTION

Graphene silicone is a material which is mixture of graphene and silicone which can radiate far infrared. The oscillation frequency from the graphene is near to that of water which is a main component of human body. As a result it has the effect of increasing body temperature, and activation of cells.

However, conventional graphene silicone is chemically inertial, steady and hydrophobic. It is very difficult to react with other molecule. Therefore, general graphene silicone cannot adhere with other objects. As a result, the application thereof is limited greatly.

Therefore, the inventor of the present invention desire a provide a novel graphene silicone sticky sheet which is adhesive with other objects, such as clothes so that the far infrared can emit to the skin of the user wearing the clothes. Thereby, the water in human body may oscillate efficiently so as to increase the circulation of human body and thus improve the health of the human.

### SUMMARY OF THE INVENTION

Accordingly, the object of the present invention is to provide a graphene silicone sticky sheet and compound structure using the same, wherein the graphene silicone with PU function group is used as substrate which is further combined with an adhering layer so as to form as a graphene silicone sticky sheet. Therefore, it is adhered to various application objects, especially clothes or wearing objects, such as an eye mask. When the user wears a clothes or a wearing object with the graphene silicone sticky sheet, since the graphene silicone in the graphene silicone sticky sheet has the function of emission of far infrared which oscillatse with water in the human body. Therefore, the temperature of human body is increased and blood circulation is promoted. Furthermore the graphene silicone sticky sheet is skin friendly as it contacts with the skin of human, the skin will feel comfortable. Further, the graphene silicone of the present invention can be connected to a conductive wire which extends outwards and thus it can be made as electrode adhesive sheet or electric heating sheet. By the skin friendly property of the silicone and the properties of having no allergic reaction, the graphene is very close to the water of human body so as to have optimum oscillation effect of water under the condition of same far infrared. By the graphene silicone sticky sheet matching to the circulation system of human body, the graphene silicone sticky sheet can be worn on a part of the human body to generate a very useful effect.

To achieve above object, the present invention provides a graphene silicone sticky sheet; the graphene silicone sticky sheet being a soft piece like or plate like structure; comprising: a graphene silicone layer formed of material which is mixture of graphene and silicone, and an interfacing layer on the graphene silicone layer; the interfacing layer being formed by material containing PU functional group material,; material containing PU (polyurethane) functional group meaning material containing isocyanate functional group and polyol; and an adhering layer arranged on the interface layer and having a soft piece like or plate like structure; and the adhering layer being is sticky and adhesive to any object.

The above sticky sheet can be added with a cloth layer adhering to the adhering layer so as to be made as clothe and eye masks. The clothes may be one of heat generated clothes, shapewear and panties.

Furthermore, a conductive layer within the interfacing layer; wherein the graphene silicone layer being formed with electric joint one end of which is connected to the conductive layer; another end of the electric joint is connected to an external conductive wire which is extended outwards for being connected to an external power source. A retaining sheet is connected to the adhering layer of the graphene silicone sticky sheet; and wherein in use, the graphene silicone layer is contacted with skins of users; when the conductive wires receive power from external power source; current will flow through the graphene silicone layer so that the graphene silicone layer stimulates user’s skin to have the effect of massage.

Furthermore, the interface layer of the graphene silicone sticky sheet may have conductive wires; one ends of the conductive wires extend out of the graphene silicone sticky sheet to be connected to an external power source; and a cloth layer adhered to the adhering layer of the graphene silicone sticky sheet; when the conductive wire is electrically conductive and thus emitted heat; the heat disperses to the whole graphene silicone layer so as to heat the cloth layer.

### BRIEF DESCRIPTION OF THE DRAWINGS

FIG. 1 is an assembly schematic view of the element of the graphene silicone sticky sheet of the present invention.

FIG. 2 is a cross section schematic view for another application of the graphene silicone sticky sheet according to the present invention.

FIG. 3A shows the first application of the second embodiment of the present invention.

FIG. 3B is a schematic view along line B-B′of FIG. 3A.

FIG. 4A shows the second application of the second embodiment of the present invention.

FIG. 4B is a schematic view along line C-C′of FIG. 4A.

FIG. 5A is a schematic view showing the third embodiment of the present invention.

FIG. 5B is a schematic view along line D-D′of FIG. 5A.

FIG. 6A is a schematic view showing the fourth embodiment of the present invention.

FIG. 6B is a schematic view along line E-E′of FIG. 6A.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

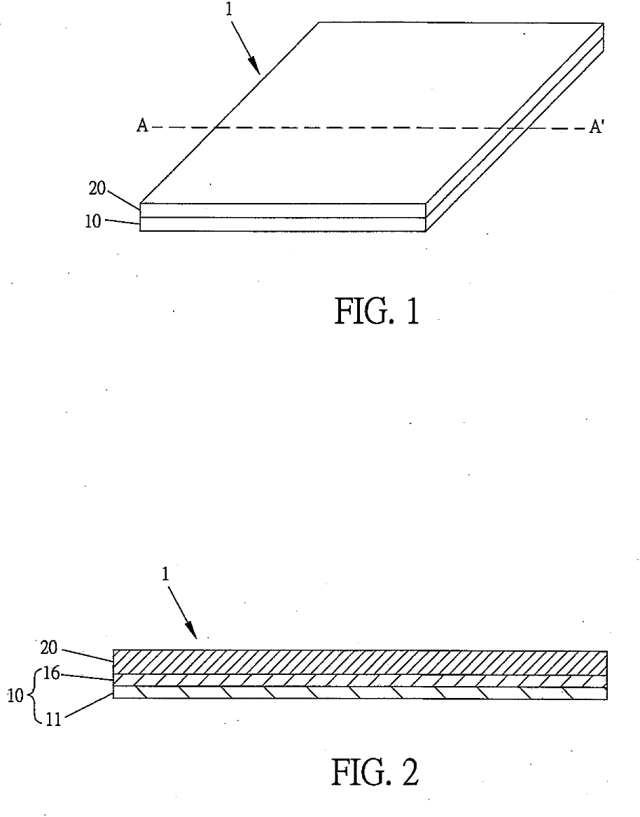

Referring to FIGS. 1 and 2, the graphene silicone sticky sheet 1 of the present invention is illustrated. The graphene silicone sticky sheet 1 of the present invention contains the following elements.

A compound silicone layer 10 of the present invention has a soft piece like or plate like structure. The compound silicone layer 10 contains a graphene silicone layer 11 (referring to FIG. 2A) and an interfacing layer 16. The graphene silicone layer 11 is formed of material which is mixture of graphene and silicone, and the interfacing layer 16 is formed by material containing PU functional group material, as illustrated in FIG. 2. Material containing PU (polyurethane) functional group means material containing isocyanate functional group and polyol. The material containing UP functional group is such as: Poly (methyl 2-mthylpropenoate) with PU functional group which means Poly (methyl 2-mthylpropenoate) with function group of Isocyanate and polyol. The Poly (methyl 2-mthylpropenoate) is PMMA (poly (methyl methacrylate), or methyl methacrylate resin). It is generally called as acrylate.

An adhering layer 20 is arranged about the compound silicone layer 10 and has a soft piece like or plate like structure. The adhering layer 20 is sticky and is adhesive to any object.

In the present invention, the material of the adhering layer 20 may be any hot melting adhesive (HMA), press sensitive glue, reactive adhesive glue, Polyurethane Reactive (PUR), etc.

Generally, the graphene silicone cannot be adhesive to cloth, while in the present invention, by the adhesive layer 20, the graphene silicone layer 11 can be adhesive to a cloth. The silicone is highly skin friendly. Therefore, when the compound silicone layer 10 of the present invention is in contact to the skin, it has no allergic reactions. Especially, the skin of the stomach of the body is more sensitive. Therefore as the compound silicone layer 10 of the present invention is in contact with skin, the skin will feel comfortable.

The emission rate of far infrared of the graphene silicone layer 11 of the present invention is 0.916, which is far highly than other prior arts with graphene silicone layer. Therefore, by oscillation of the far infrared of the graphene silicone layer 11 of the present invention and water in human body, body temperature can be increased so as to enhance blood circulation to have the effects of release tension of people and increment of metabolism. It is also helpful to increase perspiration of human body and thus to release harmful material of human body, and is beneficial to the skin and has the effect of reduction of weight.

The graphene silicone layer 11 of the present invention can be adhesive to another function layer by the adhering layer 20 thereof so as to form as a multilayer compound structure which can be adhered to various objects.

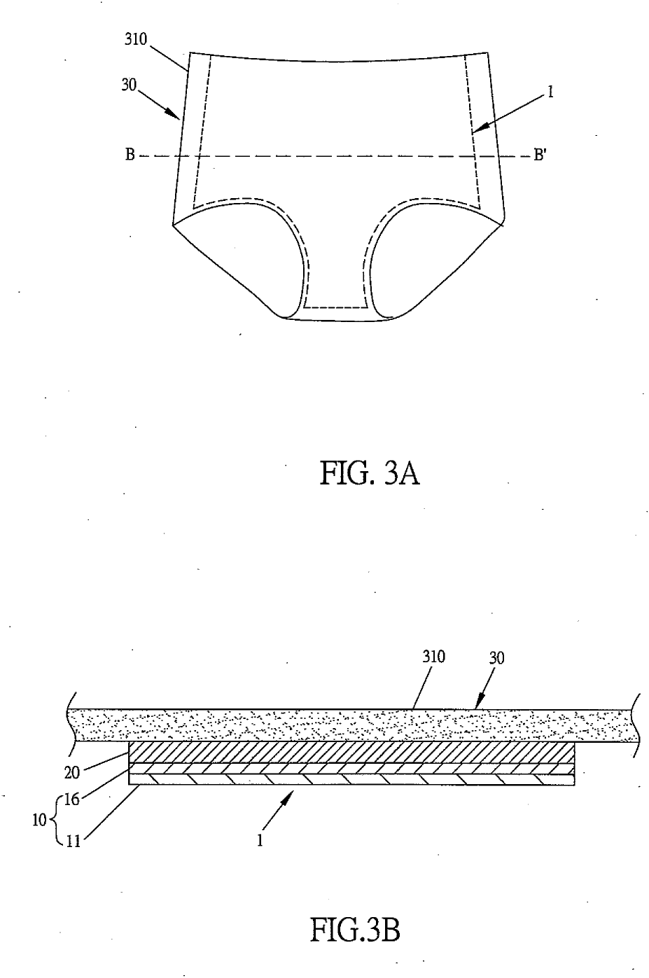

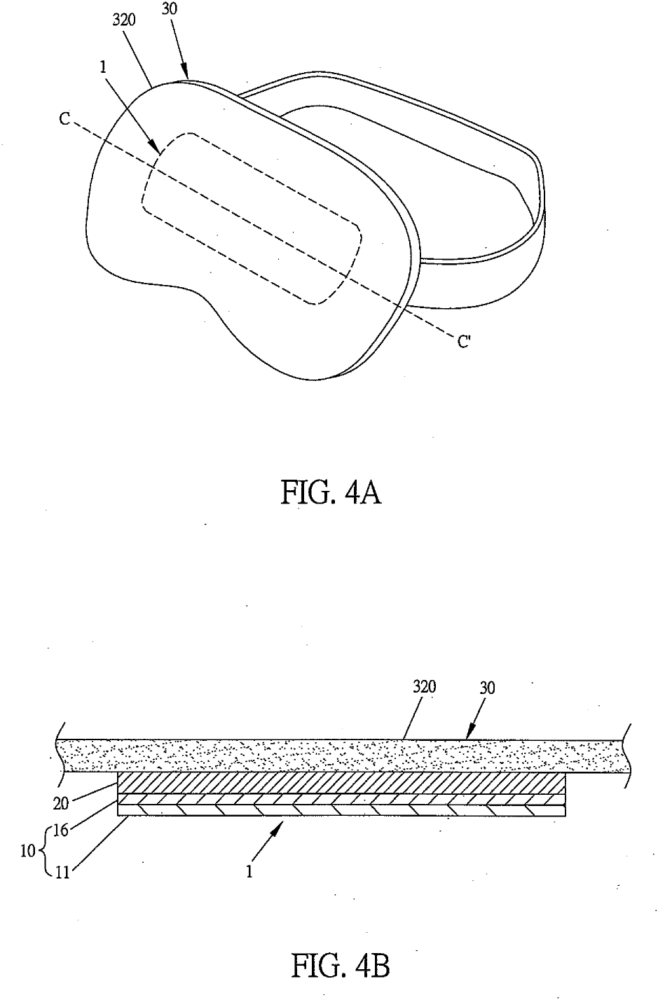

Referring to FIGS. 3A to 4B, the second embodiment of the present invention is illustrated. In this embodiment, same elements are indicated by the same numeral. In this embodiment, the graphene silicone sticky sheet 1 is combined with a material layer so as to form as a graphene silicone compound structure comprising the following elements.

A graphene silicone layer 11 as above is included.

A cloth layer 30 is adhesive to the adhering layer 20 of the graphene silicone sticky sheet. The cloth layer 30 may be a clothes or an eye mask.

Referring to FIGS. 3A and 3B shows the first application of the graphene silicone compound structure of the present invention. In that, the cloth layer 30 is clothes. Preferably, the cloth of the clothes is knitted fabric. As it is worn on the body, the graphene silicone layer 11 of the graphene silicone sticky sheet 1 face to the skin of the person wearing the clothes. Herein the clothe 310 is suitable to be made as a heat generated clothes, such as a shapewear or panties.

Preferably, when the clothes 310 is made as a panties, as illustrated in FIG. 3A, the graphene silicone layer 11 should be at a position corresponding to the lower abdomen of the user. Therefore, when the user is a woman, the position of the graphene silicone layer 11 is corresponding to the womb of the girl so as to have the effects of keeping warm and increasing blood circulation.

FIGS. 4A and 4B show the second application of the graphene silicone compound structure of the present invention, wherein the cloth layer 30 is an eye mask 320. Preferably, the cloth is knitted fabric. The eye mask 320 is worn on the eye portion of the user. The adhering layer 20 of the graphene silicone sticky sheet 1 is adhered to an inner side of the eye mask 320 corresponding to the eyes of the user. As worn on the face of the user, the graphene silicone layer 11 faces to the skin of the user.

Referring to FIGS. 3B and 4B, it is illustrated that the compound silicone layer 10 includes the graphene silicone layer 11 and an interfacing layer 16 which is the material containing PU functional group.

The graphene silicone layer 11 of graphene silicone sticky sheet 1 serves to heat eyes and the upper end of nose of the user so as to have the effect of keeping warm and improvement of blood circulation. In sleeping, this eye mask of the present invention is helpful to promote quality of sleep.

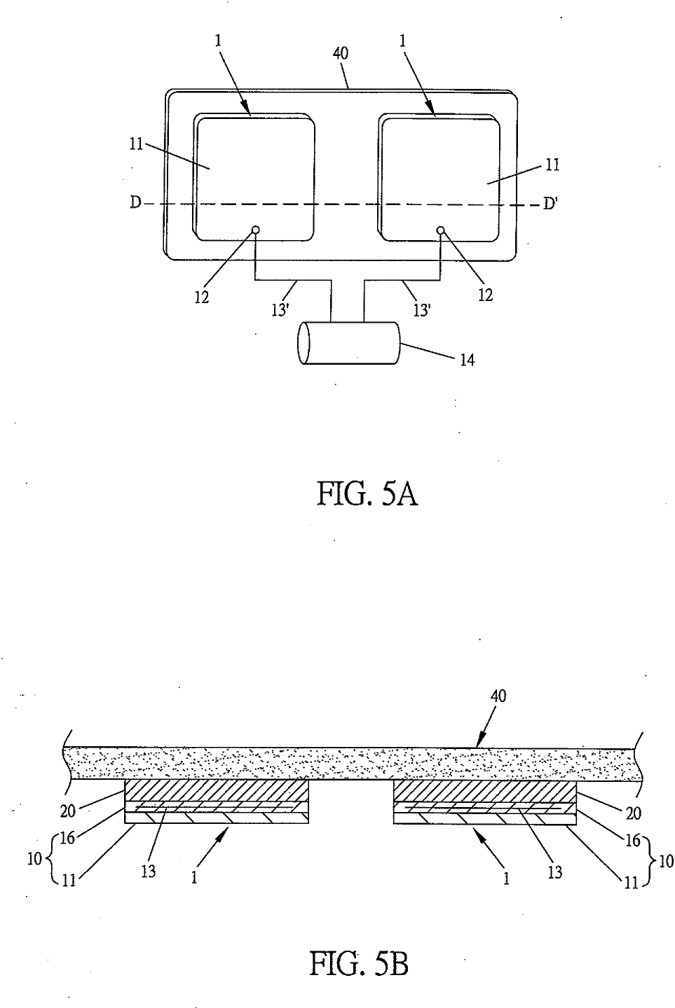

FIGS. 5 and 5B shows the third embodiment of the present invention. In this embodiment, the elements identical to those in above embodiments are illustrated by the same numerals. In this the present invention, the graphene silicone sticky sheet 1 is combined with electric conductive wires so as to form as a graphene silicone conductive compound structure. The structure contains the following elements.

Two graphene silicone sticky sheets 2 are included. The interface layer 16 of each graphene silicone sticky sheet has a conductive layer 13. The graphene silicone layer 11 of each graphene silicone sticky sheet 1 is formed with electric joint 12 one end of which is connected to the conductive layer 13. Another end of the electric joint 12 is connected to an external conductive wire 13′which is extended outwards for being connected to an external power source 14. FIG. 5B shows the conductive layer 13 is embedded into the interface layer 16.

A retaining sheet 40 is connected to the adhering layer 20 of the graphene silicone sticky sheet, as illustrated in FIG. 5A.

This application can be used as massage electric polarities, for example, as polarities in flexible hybric electronics. In use, the graphene silicone layer 11 is contacted with skins of users. When the conductive wires 13 receive power from external power source. Current will flow through the graphene silicone layer 11 of the graphene silicone sticky sheet 1 so that the graphene silicone layer 11 will stimulate user’s skin to have the effect of massage.

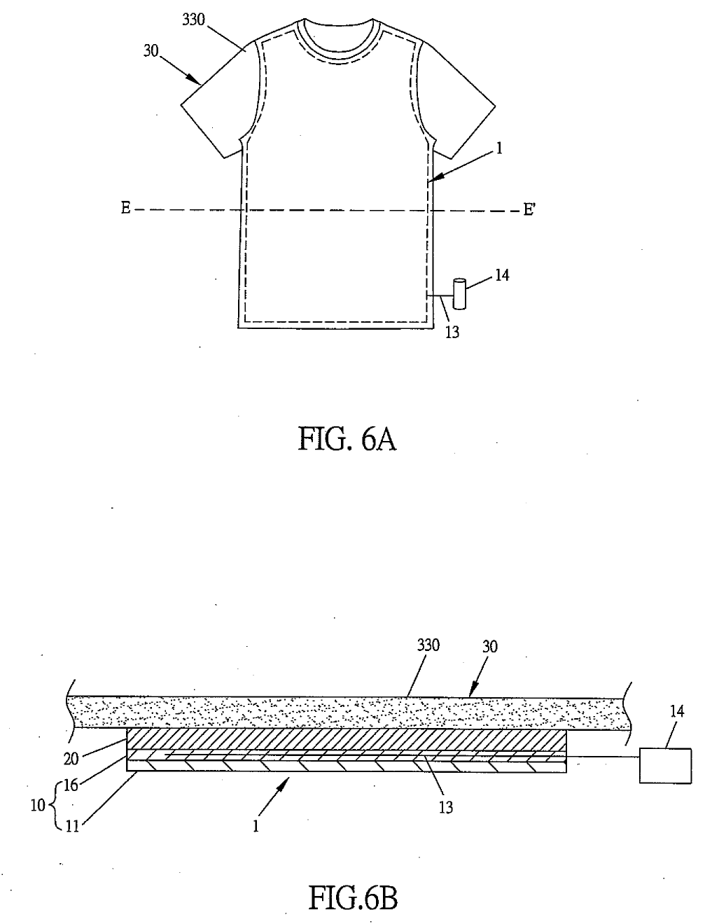

FIGS. 6A and 6B show the fourth embodiment of the present invention, in that the same elements identical to above embodiments are illustrated in the same numerals. In this embodiment, the elements identical to those in above embodiments are illustrated by the same numerals. In this the present invention, the graphene silicone sticky sheet 1 is combined with electric conductive wires so as to form as a graphene silicone conductive compound structure. The structure contains the following elements.

One graphene silicone sticky sheets 2 is included. The interface layer 16 of the graphene silicone sticky sheet has conductive wires 13. One ends of the conductive wires 13 extend out of the graphene silicone sticky sheet 1 to be connected to an external power source 14. Referring to FIG. 6B, the interface layer 16 is on an upper side of the graphene silicone layer 11. The conductive layer 13 is embedded into the interface layer 16.

A cloth layer 30 is adhered to the adhering layer 20 of the graphene silicone sticky sheet 1. The cloth layer 30 may be a clothes, preferably, it is a knitted fabric. In wearing state, the graphene silicone layer 11 of the graphene silicone sticky sheet 1 faces to the skin of a user.

When the conductive wire 13 is electrically conductive and thus emitted heat. The heat will disperse to the whole graphene silicone layer 11 so as to heat the cloth layer 30. Therefore, this application can be used as electric heating sheet. Other than the effect of uniform heat distribution, it also has the effect of electric heating which is directive, where the temperature difference connected the graphene silicone layer 11 and the temperature increment of the clothes 330 can achieve 5 degree C.

Advantages of the present invention are that the graphene silicone with PU function group is used as substrate which is further combined with an adhering layer 20 so as to form as a graphene silicone sticky sheet. Therefore, it is adhered to various application objects, especially clothes or wearing objects, such as an eye mask. When the user wears a clothes or a wearing object with the graphene silicone sticky sheet, since the graphene silicone in the graphene silicone sticky sheet has the function of emission of far infrared which oscillatse with water in the human body. Therefore, the temperature of human body is increased and blood circulation is promoted. Furthermore the graphene silicone sticky sheet is skin friendly as it contacts with the skin of human, the skin will feel comfortable. Further, the graphene silicone of the present invention can be connected to a conductive wire which extends outwards and thus it can be made as electrode adhesive sheet or electric heating sheet. By the skin friendly property of the silicone and the properties of having no allergic reaction, the graphene is very close to the water of human body so as to have optimum oscillation effect of water under the condition of same far infrared. By the graphene silicone sticky sheet matching to the circulation system of human body, the graphene silicone sticky sheet can be worn on a part of the human body to generate a very useful effect.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A graphene silicone sticky sheet; the graphene silicone sticky sheet being a soft piece like or plate like structure; comprising:
a graphene silicone layer formed of material which is mixture of graphene and silicone, and
an interfacing layer on the graphene silicone layer; the interfacing layer being formed by material containing PU functional group material,; material containing PU (polyurethane) functional group meaning material containing isocyanate functional group and polyol.

2. The graphene silicone sticky sheet as claimed in claim 1, further comprising:
an adhering layer arranged on the interface layer and having a soft piece like or plate like structure; and the adhering layer being is sticky and adhesive to any object.

3. The graphene silicone sticky sheet as claimed in claim 1, wherein the material containing UP functional group is Poly (methyl 2-mthylpropenoate) with PU functional group which means Poly (methyl 2-mthylpropenoate) with function group of Isocyanate and polyol; and the Poly (methyl 2-mthylpropenoate) is PMMA (poly (methyl methacrylate), or methyl methacrylate resin).

4. The graphene silicone sticky sheet as claimed in claim 2, wherein the material of the adhering layer is selected from hot melting adhesives (HMAs), press sensitive glues, reactive adhesive glues, and Polyurethane Reactive (PUR).

5. The graphene silicone compound structure, comprising:
a graphene silicone layer formed of material which is mixture of graphene and silicone, and
an interfacing layer on the graphene silicone layer; the interfacing layer being formed by material containing PU functional group material,; material containing PU (polyurethane) functional group meaning material containing isocyanate functional group and polyol; and
an adhering layer arranged on the interface layer and having a soft piece like or plate like structure; and
a cloth layer adhering to the adhering layer.

6. The graphene silicone compound structure as claimed in claim 5, wherein the material containing UP functional group is Poly (methyl 2-mthylpropenoate) with PU functional group which means Poly (methyl 2-mthylpropenoate) with function group of Isocyanate and polyol; and the Poly (methyl 2-mthylpropenoate) is PMMA (poly (methyl methacrylate), or methyl methacrylate resin).

7. The graphene silicone compound structure as claimed in claim 5, wherein the cloth layer is clothes, as it is worn on human body, the graphene silicone layer of the graphene silicone sticky sheet faces to skins of a person wearing the clothes.

8. The graphene silicone compound structure as claimed in claim 5, wherein the clothe is one of heat generated clothes, shapewear and panties.

9. The graphene silicone compound structure as claimed in claim 5, wherein the clothes is panties, the graphene silicone layer is at a position corresponding to the lower abdomen of a user; therefore, when the user is a woman, the position of the graphene silicone layer is corresponding to the womb of the woman.

10. The graphene silicone compound structure as claimed in claim 5, wherein the cloth layer is an eye mask; the eye mask is worn on eye portions of a user; the adhering layer is adhered to an inner side of the eye mask corresponding to eyes of the user; as worn on a face of the user, the graphene silicone layer faces to the skin of the user.

11. (canceled)

12. (canceled)

13. (canceled)

14. A graphene silicone conductive assembly, comprising:
a graphene silicone layer formed of material which is mixture of graphene and silicone, and
an interfacing layer on the graphene silicone layer; the interfacing layer being formed by material containing PU functional group material,; material containing PU (polyurethane) functional group meaning material containing isocyanate functional group and polyol; and
an adhering layer arranged on the interface layer and having a soft piece like or plate like structure;
wherein the interface layer of the graphene silicone sticky sheet has conductive wires; one ends of the conductive wires extend out of the graphene silicone sticky sheet to be connected to an external power source; and
a cloth layer adhered to the adhering layer of the graphene silicone sticky sheet; when the conductive wire is electrically conductive and thus emitted heat; the heat disperses to the whole graphene silicone layer so as to heat the cloth layer.
